(19) United States
(12) Patent Application Publication
(10) Pub. No.: US 2008/0026086 A1
(43) Pub. Date: Jan. 31, 2008

(54) Synergistic Neuroprotective, Combinations Of Flavanols, Hydroxystilbenes, Flavanones, Flavones, Flavonols, Proanthocyanidins And Anthocyanidins

(76) Inventors: Caroline Dawn Holyoak, Shambrook (GB); Karen Thethi, Leeds (GB)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(21) Appl. No.: 10/585,364

(22) PCT Filed: Dec. 9, 2004
(86) PCT No.: PCT/EP04/14072

§ 371 Date: Jun. 18, 2007

(30) Foreign Application Priority Data
Jan. 7, 2004 (EP) 04250039.7

(51) Int. Cl. A61K 31/343; A23L 1/30; A61K 36/15; A61P 25/28; A23L 2/00

(52) U.S. Cl. 424/770; 426/590; 426/648; 514/456

(57) ABSTRACT

A synthetic composition is provided comprising (a) one or more compounds selected from the group consisting of flavanols and hydroxystilbenes; and (b) one or more compounds selected from the group consisting of flavanones, flavones, flavonols, proanthocyanidins and anthocyanidins. Also provided is a method of reducing neuronal degeneration in an individual which method comprises administering to the individual said composition.

### FIELD OF THE INVENTION

The present invention relates to composition comprising combinations of polyphenols and their use in reducing neuronal degeneration in the hippocampus.

### BACKGROUND TO THE INVENTION

Public awareness and concerns about a number of disease states which exhibit age related decline in cognitive function have increased in recent years. These conditions are known by a number of names including dementia, senility, age-related amnesia and Alzheimer's syndrome. Although research is ongoing worldwide for pharmaceutical agents which delay or halt the decline in cognitive function, there remains a risk that the associated physiological changes are partially or totally irreversible. Therefore, there would be a clear benefit if an agent or agents could be found, preferably derivable from natural products, which could be administered by way of the normal human diet or supplementary thereto, which could have at least some prophylactic or therapeutic activity in respect of these conditions. Such agents could, for example, be used from an early age before cognitive decline presents clinically.

### SUMMARY OF THE INVENTION

We have now found that particular combinations of classes of polyphenols exhibit synergistic activity in assays indicative of an ability to delay, reduce or prevent age related decline in cognitive function.

Accordingly, in a first aspect, the present invention provides A synthetic composition comprising (a) one or more compounds selected from the group consisting of flavanols and hydroxystilbenes; and (b) one or more compounds selected from the group consisting of flavanones, flavones, flavonols, proanthocyanidins and anthocyanidins with the proviso that a combination of (i) catechin and quercetin and (ii) EGCg, pine extract and grape extract is specifically excluded;
wherein the molar ratio of total group (a) compound(s) to total group (b) compound(s) in said composition is at least 2.5:1.

As used herein, the term “synthetic composition” means a composition which is not per se found in nature and is at least in part modified from a natural source and/or at least partially produced by synthetic organic chemistry.

Preferably (a) comprises one or more flavanols and (b) comprises one or more flavonols.

The present invention also provides a medicament or nutritional supplement comprising a composition of the invention together with a pharmacologically acceptable carrier. The present invention further provides a food product comprising a composition of the invention and a beverage comprising a composition of the invention.

In a second aspect, the present invention provides a method of reducing neuronal degeneration in an individual which method comprises administering to the individual, an effective amount of a composition which comprises (a) one or more compounds selected from the group consisting of flavanols and hydroxystilbenes; and (b) one or more compounds selected from the group consisting of flavanones, flavones, flavonols, proanthocyanidins and anthocyanidins. Also provided is the use of said composition in the manufacture of a medicament for use in reducing neuronal degeneration in an individual.

In a third aspect, the present invention provides a method of delaying, reducing or preventing an age-related decline in cognitive function in a mammal such as human which method comprises administering to the mammal an effective amount of a composition which comprises (a) one or more compounds selected from the group consisting of flavanols and hydroxystilbenes; and (b) one or more compounds selected from the group consisting of flavanones, flavones, flavonols, proanthocyanidins and anthocyanidins. Also provided is the use of said composition in the manufacture of products capable of delaying, reducing or preventing an age related decline in cognitive function in a mammal such as a human.

### DETAILED DESCRIPTION OF THE INVENTION

Unless defined otherwise, all technical and scientific terms used herein have the same meaning as commonly understood by one of ordinary skill in the art.

Compositions according to the present invention comprise one or more compounds of group (a) as hereinbefore defined and one or more compounds of group (b) as hereinbefore defined. Preferably, the total molar ratio of group (a) compound(s) to group (b) compound(s) is at least 2.5:1, more preferably at least 3:1, more preferably at least 4:1. Preferably, the total molar ratio of group (a) compound(s) to group (b) compound(s) is less than 50:1.

Compounds of group (a) are selected from flavanols and hydroxystilbenes. Flavanols include catechin, epicatechin, gallocatechin, epigallocatechin, and esters thereof with gallic acid, i.e. catechin gallate epicatechin gallate, gallocatechin gallate and epigallocatechin gallate (EGCg). Preferred flavanols have one or more gallate moieties. Hydroxystilbenes include resveratrol and oxyresveratrol.

The compounds can be chemically synthesised or obtained from plant materials. For example, epigallocatechin gallate can be obtained from green tea extract.

Compounds of group (b) are selected from flavanones, flavones, flavonols, proanthocyanidins and anthocyanidins.

Flavanones include naringenin, hesperitin sakranetin. Flavones include luteolin and apigenin. Flavonols include quercitin, kaempferol and myricetin. Flavonols exist in nature as the aglycone or as O-glycosides. Glycosylated forms are preferred.

Again, the compounds can be chemically synthesised or obtained from plant materials. For example, in a preferred embodiment, a mixture of proanthocyanidins and anthocyanidins is provided as an extract of the bark of French maritime pine (Pinus pinatus). One such extract is available commercially as Pycnogenol™.

A plant extract differs from the intact plant material in that the various components present in the intact plant material will be present in different amounts in the extract, or substantially absent. Prior to extraction, plant materials may be dried and or mechanically processed, e.g. crushed.

Extracts of plant materials are typically made by solvent extraction. Solvents include “solvent” includes polar and non-polar organic solvents, water, and mixtures thereof. Preferred solvents are water, ethanol and mixtures thereof. Extraction procedures may include a heating step. Solvent extracted components may be subject to further purification/separation steps such as chromatography or fractional distillation. As used herein, “fraction” means any fractioned part of a solvent containing one or more of the active ingredients described above, e.g. obtained by chromatography or by fractional distillation.

Suitable plant sources of the various polyphenolic compounds described above include tea, fresh fruit such as grapes (skin and seeds in particular), cranberry, blackcurrants, blackberries and citrus fruits, and vegetables such as onions, kale, broccoli and French beans.

The solubility of flavonols in aqueous solvents can be increased by co-dissolving one or more anthocyanidins (see U.S. Pat. No. 6,569,446).

In a preferred embodiment, the composition comprises one or more flavanols. Preferably, the composition comprises a flavanol gallate ester, more preferably EGCg.

In another preferred embodiment, the composition comprises one or more flavonols. Preferably, the composition comprises myricetin and/or quercetin, more preferably quercetin.

In another embodiment (b) comprises a mixture of proanthocyanidins and anthocyanidins provided as a plant extract, such as an extract of French maritime pine bark.

Other compounds in group (a) and group (b) and/or plant extracts containing the same can be tested to confirm that possess suitable activity using, e.g, the assay methods described in the examples.

The compositions may optionally further include vitamin C and/or vitamin E.

### Compositions and Product Forms

Compositions of the invention can be combined with a pharmaceutically acceptable carrier or diluent to produce a pharmaceutical composition or nutritional supplement. Pharmaceutically acceptable diluents or carriers suitable for use in such compositions are well known in the art of pharmacy. The compositions of the invention typically contain from 0.002 to 10% by weight of total active (group (a)+group (b) compounds), such as from 0.2 to 5% by weight of active, more preferably at least 1 wt % of active.

The pharmaceutical composition may consist of solid dosage forms such as tablets, hard gelatin capsules, soft gelatin capsules, bulk powders, and microcapsules of the drug. Alternately, it may consist of a liquid dosage form such as an aqueous or non-aqueous solution, emulsion, or suspension.

Solid compositions for oral administration are preferred compositions of the invention. Solid compositions of the invention are preferably prepared in unit dosage form, such as in the form of tablets and capsules. Suitably tablets may be prepared by mixing the active combination with an inert diluent such as calcium phosphate in the presence of disintegrating agents, for example maize starch, and lubricating agents, for example magnesium stearate, and tableting the mixture by known methods. Such tablets may, if desired, be provided with enteric coatings by known methods, for example by the use of cellulose acetate phthalate. Similarly, capsules, for example hard or soft gelatin capsules, containing the active combination optionally in the form of beads with or without added excipients, may be prepared by conventional means and, if desired, provided with enteric coatings in a known manner. The tablets may be formulated in a manner known to those skilled in the art so as to give a controlled release of the compound of the present invention.

Controlled release forms of the pharmaceutical compositions of the present invention include rapid release formulations such as soluble granules or melt filled fast release capsules, delayed release formulations such as tablets provided with enteric coatings, for example, of cellulose acetate phthalate and, in particular, sustained release formulations. Numerous types of sustained release formulations are known to those skilled in the art. Typically, the active combination may be encapsulated within a release retarding coating, for example, a copolymer of cellulose ether and acrylate, or may be bound to small particles such as, for example, ion exchange resin beads. Alternatively, the active combination may be incorporated into a matrix containing a release retarding agent such as a hydrophilic gum e.g. xanthan gum, a cellulose derivative eg. hydroxypropyl methylcellulose, or a polysaccharide, wax or plastics material.

The active combination may be formulated into a solid dosage form in which the active ingredients are kept separate. For example, the dosage form may be a bilayer tablet in which the active ingredients are contained in different layers. The different layers can be formulated so as to provide the optimum release profile for each drug.

Liquid fill compositions for example viscous liquid fills, liquid paste fills or thixotropic liquid fills are also suitable for oral administration. Melt filled compositions may be obtained by mixing the active combination with certain esters of natural vegetable oil fatty acids, for example, the Gelucire™ range available from Gattefosse to provide a variety of release rates. Suitably a melt-filled capsule comprises from 10 to 80% active and from 20 to 90% of a fatty acid ester excipient which comprises one or more polyol esters and triglycerides of natural vegetable oil fatty acids.

Preferably oral liquid compositions comprise from 0.2 to 10 wt % active together with from 1 to 50 wt % of a diluent, the remainder made up with sterile water. Optionally the composition may contain suspending agents, thickeners, cosolvents such as alcohol and/or preservatives. Suitable diluents include sweetening agents for example sorbitol, xylitol or sucrose. Suitable suspending agents or thickeners include cellulose gums, agar or natural gums, for example xanthan gum. Flavourings or other taste-masking agents known to those skilled in the art for example saccharin, acesulpham K or aspartame may be added.

Compositions of the invention suitable for parenteral administration can be prepared by combination of the active with known pharmaceutical forms for such administration, for example sterile suspensions or sterile solutions of the active in a suitable solvent such as saline.

The preferred mode of administration is orally.

The amount of the compound administered depends upon the bioavailability of the compound from the pharmaceutical composition, in particular where oral administration is used. Typically, however, the compounds of this invention are dosed in an amount of from about 0.1 mg/kg of body weight to about 100 mg/kg, preferably from about 1 to about 30 mg/kg of body weight. The amount of the pharmaceutical composition depends upon the percent of compound within its formula, which is a function of the amount of the compound required per dose, its stability, release characteristics and other pharmaceutical parameters. The doses are typically administered from once or twice weekly to once or twice daily.

The routes of administration and dosages described are intended only as a guide since a skilled practitioner will be able to determine readily the optimum route of administration and dosage for any particular individual.

The compositions of the invention may also be provided as a food product which does not therefore necessarily require use of a pharmacologically acceptable carrier.

As used herein, the term “food products” includes both food products as such and beverages. Suitable food products as such include spreads, dairy products (including milk and yoghurts), desserts, convenience foods/snacks, breakfast cereals and cereal bars, mayonnaises, dressings, sandwich fillings, ready-cook meals, bread and frozen confections such as ice creams, water ices and sorbets and yoghurt ice creams. Food products also include dietary/nutritional supplements. Suitable beverages include tea, tea-flavoured drinks, coffee, soft drinks (e.g. carbonated squashes etc) and fruit juice.

The food products are supplemented with the active ingredients of the invention so that they contain higher amounts of the active ingredient(s) than they would normally contain.

The food compositions of the invention typically contain from 0.002 to 10% by weight of total active (group (a)+group (b) compounds), such as from 0.2 to 5% by weight of active, more preferably at least 1 wt % of active.

### Uses

The compositions of the invention can be used to reduce neuronal degeneration in a mammal such as a human, in particular neuronal degeneration of the central nervous system as a result of the chronic effects of glucocorticoid exposure. The compositions of the invention can also be used to reduce the glutamate-mediated excitotoxic effect resulting from chronic glucocorticoid exposure. Preferably the beneficial effects of the compositions of the invention are exerted in at least the hippocampus of the individual, i.e. the compositions of the invention can be used to reduce neuronal degeneration in the hippocampus of a mammal, such as a human and/or to reduce glutamate-mediated excitotoxic effects on the neurones of the hippocampus resulting from chronic glucocorticoid exposure. The compositions of the invention can therefore be used to reduce or alleviate hippocampal atrophy, a specific symptom resulting from chronic glucocorticoid exposure.

Consequently, the compositions of the invention may be used to delay, prevent or reduce the effects of, disease states associated with an age related decline in cognitive function, e.g. dementia, Alzheimer's related disease, cerebrovascular disease, age-related cognitive impairment and depression.

The present invention will now be described further with reference to the following examples which are illustrative only and non-limiting.

### EXAMPLES

### Materials and Methods

Dexamethasone, L-glutamate and all test agents were obtained from Sigma (except Pycnogenol which was obtained from Nature's Aid, Preston, UK) and initially dissolved in ETOH to make 10 mM stock solutions. These were then used to prepare experimental concentrations. 1:1000 dilutions were made in pre-warmed media, which were mixed by vortex prior to addition to culture. Control cultures were treated with vehicle only

### Cell Cultures

PC12 cells are a rat phreochromocytoma cell line originally described by Greene and Tischler, 1976, PNAS 73: 2424. PC12 cells were cultured in a humidified atmosphere of 95% air and 5% CO2 in RPMI 1640 (Gibco) supplemented with 10% heat inactivated horse serum, 5% FBS, 120 μg/ml streptomycin and 120 U/ml penicillin.

Confluent cells were then plated in 96-well plates pre-coated with bovine serum (20 μg/ml) and poly-d-lysine (10 μg/ml) at a density of 5000/well. Cells were grown in RPMI supplemented with 0.5% serum and 50 ng/ml nerve growth factor NGF (Sigma). Half the medium was changed every 2/3 days after initial plating until cells were fully differentiated (usually between 5-7 days). Use of NGF allows differentiation of the neurons to a sympathetic phenotype-expressing neurites and excitability (Fujita, 1989, Environ. Health Perspect. 80:127-42).

All media was then removed and 10 μM dexamethasone in fresh media was added to the cells for 72 hours (to mimic chronic stress). This was removed and replaced by the test micronutrient actives which were left on the cells for 24 hours. 1 mM L-glutamate was then added to the cells (to mimic an acute stress) and cell supernatant assayed for PGE₂, LTB₄ using commercial ELISA kits (Amersham Pharmacia Biotech APB) and cell viability using the MTT assay (Sigma).

### Mitochondrial Succinate Dehydrogenase Assay (MTT)

MTT reduction assay is one of the most widely used assays for determining cell viability (Shearman et al, 1994, PNAS USA. 91:1470-1474; Liu and Schubert 1997, J Neurochem 69: 2285-93; Liu et al, 1997, J. Neurochem. 69: 581-93), it detects living but not dead cells and the signal generated is dependent upon the degree of activation of the cells (Mosmann, 1983, J. Immunol Methods. 65: 55-63). A modified MTT assay was used. Briefly 10 μl of a 5 mg/ml MTT solution in sterile PBS was added to 100 μl of medium and incubated for 2-4 hours at 37° C. The reaction was stopped using 100 μl of isopropanol. The plates were then vigorously shaken at room temperature for a further 2 hours until the formazan crystals had fully dissolved. Absorbance readings at 570 nm were then taken. Results of MTT reduction in treated cells were expressed as a percentage of control (untreated cells). All the experiments were performed in triplicate.

Prior to the performing the ELISA assays for PGE₂ and LTB₄ and the cell viability assays 10 μM arachadonic acid (Sigma) was added to each microtitre plate well this was followed 5 minutes later by 2 μM calcium ionophore A23187 (Sigma). Plates were then left at room temperature for a further 15 minutes.

### Prostaglandin E₂

The Biotrak™ prostaglandin E₂ (PGE₂) competitive enzyme immunoassay was used to determine levels of PGE₂ in cell supernatants. The assay was carried out according to the manufacturer's instructions using triplicate samples.

### Leukotriene B₄

The Biotrak™ Leukotriene B₄ (LTB₄) enzyme immunoassay was used to determine levels of LTB₄ in cell supernatants. The assay was carried out according to the manufacturer's instructions using triplicate samples.

Experiments were run using triplicate wells on a 96-well plate and each experiment was repeated three times unless otherwise stated. Statistical analyses of the effects of treatments versus controls were calculated using t-tests.

Pairs of agents were tested to determine whether they gave a synergistic reduction in prostaglandin E₂ (PGE₂) and leucotriene B₄ (LTB₄) in vitro. Dunnet's test was used to determine whether the results obtained indicated synergy.

### Results

In the following table, the results are codified as:
+ 1-5% synergistic reduction
++ >5-10% synergistic reduction
+++ >10-20% synergistic reduction
++++ >20-30% synergistic reduction
+++++ >30% synergistic reduction
NS—not significantly different from either treatment administered individually.

| Treatment 1 | Treatment 2 | PGE₂ | LTB₄ |
|---|---|---|---|
| 0.25 μM Quercetin | 0.25 μM EGCg | + | ++ |
| | 0.50 μM EGCg | + | ++ |
| | 1.0 μM EGCg | ++ | ++ |
| | 2.0 μM EGCg | +++ | ++ |
| 0.25 μM Quercetin | 0.25 μM Resveratrol | + | + |
| | 0.50 μM Resveratrol | + | ++ |
| | 1.0 μM Resveratrol | + | ++ |
| | 2.0 μM Resveratrol | ++ | ++ |
| 0.25 μM Quercetin | 1.0 μM Epicatechin | + | + |
| | 2.0 μM Epicatechin | ++ | + |
| 0.25 μM Quercetin | 1.0 μM Gallic Acid | + | + |
| | 2.0 μM Gallic Acid | + | + |
| 0.50 μM Quercetin | 0.25 μM EGCg | + | ++ |
| | 0.50 μM EGCg | + | ++ |
| | 1.0 μM EGCg | ++ | ++ |
| | 2.0 μM EGCg | +++ | ++ |
| 0.50 μM Quercetin | 0.25 μM Resveratrol | + | ++ |
| | 0.50 μM Resveratrol | + | ++ |
| | 1.0 μM Resveratrol | ++ | +++ |
| | 2.0 μM Resveratrol | +++ | +++ |
| 0.50 μM Quercetin | 0.25 μM Epicatechin | + | + |
| | 0.50 μM Epicatechin | + | + |
| | 1.0 μM Epicatechin | ++ | ++ |
| | 2.0 μM Epicatechin | ++ | ++ |
| 0.50 μM Quercetin | 0.50 μM Gallic Acid | + | + |
| | 1.0 μM Gallic Acid | + | + |
| | 2.0 μM Gallic Acid | ++ | ++ |
| 1.0 μM Quercetin | 0.25 μM EGCg | + | +++ |
| | 0.50 μM EGCg | + | ++++ |
| | 1.0 μM EGCg | +++ | +++++ |
| | 2.0 μM EGCg | +++ | +++++ |
| 1.0 μM Quercetin | 0.25 μM Resveratrol | + | ++ |
| | 0.50 μM Resveratrol | ++ | ++ |
| | 1.0 μM Resveratrol | +++ | ++++ |
| | 2.0 μM Resveratrol | +++ | ++++ |
| 1.0 μM Quercetin | 0.25 μM Epicatechin | + | ++ |
| | 0.50 μM Epicatechin | ++ | ++ |
| | 1.0 μM Epicatechin | +++ | +++ |
| | 2.0 μM Epicatechin | +++ | +++ |
| 1.0 μM Quercetin | 0.25 μM Gallic Acid | + | + |
| | 0.50 μM Gallic Acid | + | + |
| | 1.0 μM Gallic Acid | ++ | + |
| | 2.0 μM Gallic Acid | ++ | ++ |
| 2.0 μM Quercetin | 0.25 μM EGCg | +++ | ++++ |
| | 0.50 μM EGCg | +++ | ++++ |
| | 1.0 μM EGCg | +++++ | +++++ |
| | 2.0 μM EGCg | +++++ | +++++ |
| 2.0 μM Quercetin | 0.25 μM Resveratrol | +++ | ++++ |
| | 0.50 μM Resveratrol | +++ | ++++ |
| | 1.0 μM Resveratrol | ++++ | +++++ |
| | 2.0 μM Resveratrol | +++++ | +++++ |
| 2.0 μM Quercetin | 0.25 μM Epicatechin | ++ | ++ |
| | 0.50 μM Epicatechin | ++ | ++ |
| | 1.0 μM Epicatechin | +++ | +++ |
| | 2.0 μM Epicatechin | +++ | +++ |
| 2.0 μM Quercetin | 0.25 μM Gallic Acid | + | ++ |
| | 0.50 μM Gallic Acid | ++ | ++ |
| | 1.0 μM Gallic Acid | ++ | +++ |
| | 2.0 μM Gallic Acid | ++ | +++ |
| 0.25 μM EGCg | 0.25 μM Myricetin | + | + |
| | 0.50 μM Myricetin | + | + |
| | 1.0 μM Myricetin | + | + |
| | 2.0 μM Myricetin | + | ++ |
| 0.25 μM EGCg | 0.25 μM Apigenin | + | + |
| | 0.50 μM Apigenin | + | + |
| | 1.0 μM Apigenin | + | + |
| | 2.0 μM Apigenin | + | + |
| 0.25 μM EGCg | 5.0 μml Pycnogenol | + | + |
| | 10.0 μ/ml Pycnogenol | ++ | + |
| | 25.0 μg/ml Pycnogenol | +++ | ++ |
| 0.50 μM EGCg | 0.25 μM Naringenin | + | ++ |
| | 0.50 μM Naringenin | + | ++ |
| | 1.0 μM Naringenin | + | +++ |
| | 2.0 μM Naringenin | + | +++ |
| 0.50 μM EGCg | 0.25 μM Myricetin | + | ++ |
| | 0.50 μM Myricetin | + | ++ |
| | 1.0 μM Myricetin | + | +++ |
| | 2.0 μM Myricetin | + | ++++ |
| 0.50 μM EGCg | 0.25 μM Apigenin | + | + |
| | 0.50 μM Apigenin | + | + |
| | 1.0 μM Apigenin | + | +++ |
| | 2.0 μM Apigenin | + | ++++ |
| 0.50 μM EGCg | 2.5 μg/ml Pycnogenol | + | + |
| | 5.0 μg/ml Pycnogenol | ++ | + |
| | 10 μg/ml Pycnogenol | ++ | + |
| | 25 μg/ml Pycnogenol | +++ | ++ |
| 1.0 μM EGCg | 0.25 μM Naringenin | + | ++ |
| | 0.50 μM Naringenin | + | ++ |
| | 1.0 μM Naringenin | ++ | +++ |
| | 2.0 μM Naringenin | ++ | ++++ |
| 1.0 μM EGGg | 0.25 μM Myricetin | + | ++++ |
| | 0.50 μM Myricetin | + | ++++ |
| | 1.0 μM Myricetin | ++ | +++++ |
| | 2.0 μM Myricetin | +++ | +++++ |
| 1.0 μM EGCg | 0.25 μM Apigenin | + | ++ |
| | 0.50 μM Apigenin | + | +++ |
| | 1.0 μM Apigenin | ++ | ++++ |
| | 2.0 μM Apigenin | ++ | +++++ |
| 1.0 μM EGGg | 2.5 μg/ml Pycnogenol | ++ | + |
| | 5.0 μg/ml Pycnogenol | ++++ | ++ |
| | 10 μg/ml Pycnogenol | ++++ | +++ |
| | 25 μg/ml Pycnogenol | ++++ | ++++ |
| 2.0 μμM EGCg | 0.25 μM Naringenin | + | ++ |
| | 0.50 μM Naringenin | + | +++ |
| | 1.0 μM Naringenin | ++ | +++ |
| | 2.0 μM Naringenin | +++ | ++++ |
| 2.0 μM EGCg | 0.25 μM Myricetin | + | ++++ |
| | 0.50 μM Myricetin | ++ | +++++ |
| | 1.0 μM Myricetin | +++ | +++++ |
| | 2.0 μM Myricetin | +++ | +++++ |
| 2.0 μM EGCg | 0.25 μM Apigenin | + | +++ |
| | 0.50 μM Apigenin | ++ | ++++ |
| | 1.0 μM Apigenin | +++ | +++++ |
| | 2.0 μM Apigenin | +++ | +++++ |
| 2.0 μM EGCg | 2.5 μg/ml Pycnogenol | +++ | ++++ |
| | 5.0 μg/ml Pycnogenol | +++ | ++++ |
| | 10 μg/ml Pycnogenol | ++++ | +++++ |
| | 25 μg/ml Pycnogenol | +++++ | +++++ |

### COMPARATIVE EXAMPLES

| Treatment 1 | Treatment 2 | PGE₂ | LTB₄ |
|---|---|---|---|
| 1.0 μM EGCG | 0.25 μM Resveratrol | NS | NS |
| | 0.50 μM Resveratrol | NS | NS |
| | 1.0 μM Resveratrol | NS | NS |
| | 2.0 μM Resveratrol | NS | NS |
| 0.25 μM Naringenin | 0.25 μM Quercetin | NS | NS |
| | 0.50 μM Quercetin | NS | NS |
| | 1.0 μM Quercetin | NS | NS |
| | 2.0 μM Quercetin | NS | NS |

These comparative examples demonstrate that a combination of two compounds both of which fall into group (a) (EGCg is a flavanol and resveratrol is a hydroxystilbene) does not show synergy. Equally, a combination of two compounds both of which fall into group (b) (naringenin is a flavanone and quercetinis a flavonol) does not show synergy. It is only combinations where one compound is from group (a)—flavanols and hydroxystilbenes—and one is from group (b)—flavanones, flavones, flavonols, proanthocyanidins and anthocyanidins—which show synergy.

The various features and embodiments of the present invention, referred to in individual sections above apply, as appropriate, to other sections, mutatis mutandis. Consequently features specified in one section may be combined with features specified in other sections, as appropriate.

All publications mentioned in the above specification are herein incorporated by reference. Various modifications and variations of the described methods and products of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in the relevant fields are intended to be within the scope of the following claims.

1. A synthetic composition comprising (a) one or more compounds selected from the group consisting of flavanols and hydroxystilbenes; and (b) one or more compounds selected from the group consisting of flavanones, flavones, flavonols, proanthocyanidins and anthocyanidins with the proviso that a combination of (i) catechin and quercetin and (ii) EGCg, pine extract and grape extract is specifically excluded;
wherein the molar ratio of total group (a) compound(s) to total group (b) compound(s) in said composition is at least 2.5:1.

2. A composition according to claim 1, wherein (a) comprises one or more flavanols and (b) comprises one or more flavonols.

3. A composition according to claim 1, wherein (a) comprises EGCg and (b) comprises quercetin.

4. A composition according to claim 1 wherein (b) comprises a mixture of proanthocyanidins and anthocyanidins provided as a plant extract.

5. A composition according to claim 4 wherein the plant extract is an extract of French maritime pine bark.

6. A composition according to claim 1, wherein the molar ratio of total group (a) compound(s) to total group (b) compound(s) is less than 50:1.

7. A medicament or nutritional supplement comprising a composition according to claim 1 together with a pharmacologically acceptable carrier.

8. A medicament or nutritional supplement according to claim 7, comprising from 0.002% to 10% by weight of total group (a) compound(s) plus total group (b) compound(s).

9. A food product comprising a composition according to claim 1.

10. A food product according to claim 9, comprising from 0.002% to 10% by weight of total group (a) compound(s) plus total group (b) compound(s).

11. A beverage comprising a composition according to claim 1.

12. A beverage according to claim 11, comprising from 0.002% to 10% by weight of total group (a) compound(s) plus total group (b) compound(s).

13. A method of reducing neuronal degeneration in an individual which method comprises administering to the individual, a composition which comprises (a) one or more compounds selected from the group consisting of flavanols and hydroxystilbenes; and (b) one or more compounds selected from the group consisting of flavanones, flavones, flavonols, proanthocyanidins and anthocyanidins.

14. A method according to claim 13 wherein the neuronal degeneration is in the hippocampus of the individual.

15. A method of manufacture of a medicament for use in reducing neuronal degeneration in an individual comprising use of a composition which comprises (a) one or more compounds selected from the group consisting of flavanols and hydroxystilbenes; and (b) one or more compounds selected from the group consisting of flavanones, flavones, flavonols, proanthocyanidins and anthocyanidins.

16. A method of delaying, reducing or preventing an age-related decline in cognitive function in a mammal which method comprises administering to the mammal a composition which comprises (a) one or more compounds selected from the group consisting of flavanols and hydroxystilbenes; and (b) one or more compounds selected from the group consisting of flavanones, flavones, flavonols, proanthocyanidins and anthocyanidins.

17. A method of manufacture of a product for use in delaying, reducing or preventing an age related-decline in cognitive function in a mammal comprising use of a composition which comprises (a) one or more compounds selected from the group consisting of flavanols and hydroxystilbenes; and (b) one or more compounds selected from the group consisting of flavanones, flavones, flavonols, proanthocyanidins and anthocyanidins.
